(19) United States
(12) Patent Application Publication
(10) Pub. No.: US 2024/0002783 A1
(43) Pub. Date: Jan. 4, 2024

(54) METHOD FOR STIMULATING THE GROWTH OF A CELL

(71) Applicant: 1000414206 ONTARIO INC., Toronto (CA)

(72) Inventor: John Greogry MARSHALL, Toronto (CA)

(21) Appl. No.: 18/162,807

(22) Filed: Feb. 1, 2023

Related U.S. Application Data
(63) Continuation of application No. PCT/CN2021/110473, filed on Aug. 4, 2021.
(60) Provisional application No. 63/061,328, filed on Aug. 5, 2020.

(51) Int. Cl. C12N 5/00

(52) U.S. Cl. CPC C12N 5/0018; C12N 2501/33; C12N 2501/11

(57) ABSTRACT

A method for stimulating the growth of a cell comprising contacting the cell with a composition comprising an isolated lens epidermal dermal growth factor.

### FIELD OF THE DISCLOSURE

The present invention relates to a method for stimulating the growth of a cell; more particularly, to a method for stimulating the growth of a cell with lens epidermal dermal growth factor (LEDGF).

### BACKGROUND OF THE DISCLOSURE

Fetal serum contains factors that promote changes in gene expression and permit the indefinite culture of cell lines such as RAW 264.7 murine macrophages. Insulin, nerve growth factor, epidermal growth factor, insulin-like growth factor, fibroblast growth factor (FGF), platelet-derived growth factor and transforming growth factor (TGF) have been shown to regulate cellular proliferation. However, the effect of growth hormones was inferior to that of fetal serum even after supplementation with albumin, transferin, amino acids or dilute serum (Yao, T and Y. Asayama, Animal-cell culture media: History, characteristics, and current issues. Reprod Med Biol, 2017. 16(2): p. 99-117) and so all of the factors that permit indefinite cell growth have yet to be determined (Kwon, D., et al., The Effect of Fetal Bovine Serum (FBS) on Efficacy of Cellular Reprogramming for Induced Pluripotent Stem Cell (iPSC) Generation. Cell Transplant, 2016. 25(6): p. 1025-42). Fetal serum or platelet lysates have also been shown to expand clonal cell lines and may have therapeutic applications and have basic biochemical importance (Castellano, J. M., et al., Human umbilical cord plasma proteins revitalize hippocampal function in aged mice. Nature, 2017. 544(7651): p. 488-492). There has been significant efforts to create cell growth media that contains the types of growth factors contained in natural serum for use in cell culture experiments and attempts to enumerate the proteins of fetal serum from umbilical chords or other approaches (Li, C., F. Wang, and L. Liu, Ultra-high performance liquid chromatography-mass spectrometry for analysis of newborn and fetal bovine serum components. Nan Fang Yi Ke Da Xue Xue Bao, 2014. 34(5): p. 751-3).

### SUMMARY OF THE DISCLOSURE

Lens epidermal dermal growth factor is firstly identified from fetal serum and found to stimulate the growth of a cell.

The present disclosure provides a method for stimulating the growth of a cell comprising contacting the cell with a composition comprising an isolated lens epidermal dermal growth factor.

In one embodiment of the disclosure, the composition is a purified fetal serum.

In one embodiment of the disclosure, the composition further comprises a hormone.

In one embodiment of the disclosure, the hormone is a growth related hormone.

In one embodiment of the disclosure, the hormone is selected from the group consisting of insulin, insulin like growth factor 2, multiple EGF like domains 11 (MEGF11), erythropoietin (EPO), pigment epithelium-derived factor [PEDF, also known as serpin F1 (SERPINF1)], interleukin-17D (IL 1 7D), interleukin-37 (IL37), interleukin-17C, interleukin-13, interleukin-2, retinol-binding protein 4 (RBP4), tissue factor pathway inhibitor (TFPI, preferably tissue factor pathway inhibitor 2), nerve growth factor beta polypeptide (NGFB), growth hormone somatotropin (GH1/GH), multiple EGF like domains 6 (MEGF6), tumor necrosis factor (TNF, preferably TNF alpha), cardiotrophin like cytokine factor 1 (CLCF1), teratocarcinoma-derived growth factor (TDGF, preferably teratocarcinoma-derived growth factor-1), glycoprotein hormone subunit beta 5 (GPHB5), cytokine receptor like factor 1 (CRLF1), transforming growth factor beta (TGFB, preferably transforming growth factor beta 2), growth hormone exon 5 (GHE5), C—C motif chemokine ligand 21 (CCL21), platelet derived growth factor (PDGF), platelet derived growth factor receptor like (PDGFRL), growth differentiation factor (GDF, preferably growth differentiation factor 15, 11, 6, 5 or 3), fibroblast growth factor (FGF, preferably fibroblast growth factor 18, 14 or 8), C—X—C motif chemokine ligand (CXCL, preferably C—X—C motif chemokine ligand 13, 9 or 6), and insulin like growth factor binding protein (IGFBP, preferably insulin like growth factor binding protein 4 or 2).

In one embodiment of the disclosure, the cell is a stem cell.

In one embodiment of the disclosure, the method is for stimulating more cell division cycles.

In one embodiment of the disclosure, the method is for prolonging the lifespan of the cell.

In one embodiment of the disclosure, the step of contacting the cell is in a cell culture. In one embodiment of the disclosure, the step of contacting the cell is in situ.

The present disclosure is described in detail in the following sections. Other characteristics, purposes and advantages of the present disclosure can be found in the detailed description and claims.

### BRIEF DESCRIPTION OF THE DRAWINGS

FIG. 1A show the effect of fetal calf serum on the morphology of RAW 264.7 murine macrophages cultured in DMEM plus 5% fetal calf serum; FIG. 1B show the effect of adult serum on the morphology of RAW 264.7 murine macrophages cultured in DMEM plus 5% adult bovine serum; FIG. 1C shows the distribution of cell length in adult (ABS) versus fetal (FBS) serum. Ten cells were measured from 3 spots on 3 cover slips from 3 independent batches of fetal versus adult serum. The longest axis of the cell was measured. F-statistic: 307.1 on 3 and 156 DF, p-value: <2.2e-16.

FIG. 2 shows flow chart of the protein (proteomics) and endogenous peptides (peptidomics) isolation and analysis steps.

FIG. 3A shows the fractionation of bovine plasma by a step gradient of NaCl in 20 mM Tris pH 8.85 buffer in Dumbroff protein assay; FIG. 3B shows the fractionation of bovine plasma by a step gradient of NaCl in 20 mM Tris pH 8.85 buffer. Lanes: 1, Molecular Weight Standard; 2, wash (W); 3, flow through (FT). Quaternary Amine (QA) fractions: 1, 0 mM; 2, 50 mM; 3, 100 mM; 4, 150 mM; 5, 175 mM; 6, 200 mM; 7, 225 mM; 8, 250 mM; 9, 300 mM; 10, 350 mM; 11, 400 mM; 12, 450 mM; 13, 500 mM; 14, 600 mM, 50% ACN; 16, 5% formic acid.

FIG. 4A shows the sequential extraction of low molecular mass polypeptides from serum using a stepwise solubilization in organic/water (Dumbroff protein assay); FIG. 4B shows the sequential extraction of low molecular mass polypeptides from serum using a stepwise solubilization in organic/water. Lanes: 1, Molecular Weight Standard. Acetronitrile (ACN)/water fractions 1, 90% ACN; 2, 70% ACN; 3, 60% ACN; 4, 50% ACN; 5, 40% ACN; 6, 30% ACN; 7, 20% ACN; 8, 10% ACN; 9, 5% ACN.

FIG. 5A shows the reproducibility of the fetal versus adult serum samples with the QQ plot showing the normality of the log 10 transformed intensity values; FIG. 5B shows the reproducibility of the fetal versus adult serum samples with the box plot showing the average log 10 intensity and variation in log 10 and 99% confidence interval for the adult versus fetal serum replicates. Treatments: 1, ABS QA rep1; 2, ABS QA STYP rep1; 3, ABS QA rep2; 4, ABS QA STYP rep2; 5, ABS QA rep3; 6, QA ABS QA STYP rep3; 7, ABS, ACN rep1; 8, ABS ACN STYP rep1; 9 ABS ACN rep2; 10, ABS, ACN STYP rep2; 11, ABS, ACN rep3; 12, ABS ACN STYP rep3; 13, FC QA rep1; 14, FCS QA STYP rep1; 15, FCS QA rep2; 16, FCS QA STYP rep2; 17, FCS QA rep3; 18, FCS QA STYP rep3; 19, FCS AACN rep1; 20, FCS ACN STYP rep1; 21, FCS ACN rep2; 22, FCS ACN STYP rep2; 23, FCS ACN rep3; 24, FCS ACN STYP rep3.

FIG. 6A shows the total number of protein accessions from the Bovine protein library that were correlated by the X!TANDEM and SEQUEST algorithms combined (redundant protein accessions); FIG. 6B shows the total number of protein accessions from the Bovine protein library that were correlated by the X! TANDEM and SEQUEST algorithms combined (best correlation per gene symbol). The Bovine protein

FIG. 7A shows the protein accessions identified by the SEQUEST and X!TANDEM algorithms separately. The Bovine protein library contained 209,111 protein accessions.library contained 209,111 accessions. FIG. 7B shows the protein accessions identified by the SEQUEST and X! TANDEM algorithms separately. The Bovine protein library contained 209,111 protein accessions.library contained 209,111 accessions.

FIG. 8 shows the cumulative p-value and FDR corrected q-value of the non-redundant peptides per gene symbol computed from the X! TANDEM results using the R statistical system.

FIG. 9A shows the quantile plots of the corrected difference in observation frequency (Delta) and Chi Square values of the fetal versus adult serum. Tryptic peptide corrected difference (delta) in observation frequency; FIG. 9B shows the quantile plots of the corrected difference in observation frequency (Delta) and Chi Square values of the fetal versus adult serum. Tryptic peptide Chi Square x2; FIG. 9C shows the quantile plots of the corrected difference in observation frequency (Delta) and Chi Square values of the fetal versus adult serum. Tryptic and/or STYP corrected difference (delta) in observation frequency; FIG. 9D shows the quantile plots of the corrected difference in observation frequency (Delta) and Chi Square values of the fetal versus adult serum. Tryptic and/or STYP peptide Chi Square x2.

FIG. 10 shows the effect of insulin on the longest cell axis of RAW 264.7 macrophages in Adult Bovine Serum (ABS) versus Fetal Bovine Serum (FBS). The levels of insulin added each well of a six well culture dish are shown. The letters indicate statistically significant differences by the Tukey Kramer Honestly Significant Difference (HSD) Test

FIG. 11 shows the effect of insulin on the longest cell axis of RAW 264.7 macrophages in Adult Bovine Serum (ABS) versus Fetal Bovine Serum (FBS). The levels of insulin or Lens Epithelial Derived Growth Factor (LEDGF) added each well of a six well culture dish are shown. The letters indicate statistically significant differences by the Tukey Kramer Honestly Significant Difference (HSD) Test.

### DETAILED DESCRIPTION OF THE DISCLOSURE

Unless otherwise defined herein, scientific and technical terms used in connection with the present invention shall have the meaning commonly understood by those of ordinary skill in the art. The meaning and scope of the terms should be clear; however, in the event of any latent ambiguity, definitions provided herein take precedence over any dictionary or extrinsic definition.

It must be noted that, as used in the specification and the appended claims, the singular forms “a,” “an” and “the” include plural referents unless the context clearly dictates otherwise. Thus, unless otherwise required by context, singular terms shall include the plural and plural terms shall include the singular.

As used herein, the term “or” is used to mean “and/or” unless explicitly indicated to refer to alternatives only or the alternatives are mutually exclusive.

The present disclosure provides a method for stimulating the growth of a cell comprising contacting the cell with a composition comprising an isolated lens epidermal dermal growth factor (LEDGF).

Fetal serum contains factors such as alpha fetoprotein (AFP), insulin, and insulin-like growth factor 2 that support the indefinite division and growth of cancerous cell lines. Though all of the peptides and proteins of fetal serum that regulate cell growth have yet to be elucidated. In some embodiments of the disclosure, the peptides of fetal, versus adult serum, were extracted in organic solvent and the proteins resolved on quaternary amine resin for tryptic digestions. The resulting peptides were analyzed by random and independent sampling with LC-ESI-MS/MS. Peptides and proteins were identified by the X!TANDEM and SEQUEST algorithms. Precursor and fragment intensity values with peptide and protein assignments were collected in SQL Server for analysis with the R statistical system. The 225,097 endogenous peptide sequences from peptidomics showed 23% overlap with the 431,443 distinct peptide sequences from proteomics. However the independent sets of peptides showed 99.3% agreement on a small set of protein gene symbols while most proteins were never observed. The agreement by independent methods was an unambiguous and clear demonstration that LC-ESI-MS/MS of blood plasma with a linear quadrupole ion trap shows low error rates of peptide and protein identification. Fetal serum showed increased observation frequency of peptides from proteins involved in the insulin and AKT HRAS pathways. Many growth factors including lens epithelium derived growth factor were specific to fetal serum. The addition of insulin and/or LEDGF to adult serum partially restored the rounded phenotype of rapidly dividing cells but was not as effective as fetal serum. All of the peptides and proteins in blood fluid may be enumerated in using a simple linear quadrupole ion trap with ≤1% error that revealed all the proteins specific to fetal serum that support the immortal growth of cells. Accordingly, one of the embodiment compositions is a purified fetal serum.

One ore more the hormones can be added to the composition of the present invention. In one embodiment of the disclosure, the hormone is a growth related hormone. Examples of the hormone include, but are not limited to, insulin, insulin like growth factor 2, multiple EGF like domain 11 (MEGF11), erythropoietin (EPO), pigment epithelium-derived factor (PEDF), interleukin-17D (IL17D), interleukin-37 (IL37), interleukin-17C, interleukin-13, interleukin-2, retinol-binding protein 4 (RBP4), tissue factor pathway inhibitor (TFPI2), nerve growth factor beta polypeptide (NGFB), growth hormone somatotropin (GH1/GH), multiple EGF like domain 6 (MEGF6), tumor necrosis factor (TNF), cardiotrophin like cytokine factor 1 (CLCF1), teratocarcinoma-derived growth factor (TDGF), glycoprotein hormone subunit beta 5 (GPHB5), cytokine receptor like factor 1 (CRLF1), transforming growth factor beta (TGFB), growth hormone exon 5 (GHE5), C—C motif chemokine ligand 21 (CCL21), platelet derived growth factor (PDGF), platelet derived growth factor receptor like (PDGFRL), growth differentiation factor (GDF), fibroblast growth factor (FGF), C—X—C motif chemokine ligand (CXCL), and insulin like growth factor binding protein (IGFBP).

After in vitro or in vivo contacting the composition of the present invention with a cell, the growth of the cell can be stimulated. The cell growth stimulated by the composition of the present invention includes producing more cell division cycles or prolonging the lifespan of the cell. The in vitro contacting step is performed through a cell culture process.

The following examples are provided to aid those skilled in the art in practicing the present disclosure.

### Examples

### Materials and Methods

### Materials

The HPLC was an Agilent 1100 (Santa Clara, CA, USA). The linear ion trap mass spectrometer was a LTQ XL (Thermo Electron Corporation, Waltham, MA, USA). Ceramic quaternary amine resin was from BioRad. C18 ZipTips were obtained from Millipore (Bedford, MA). C18 HPLC resin was from Agilent (Zorbax 300 SB-C18 5-micron, 300 Angstrom). Solvents were obtained from Caledon Laboratories (Georgetown, Ontario, Canada). Three independent batches of fetal calf serum (FCS) and adult bovine serum (ABS) were supplied from Cell Grow, Sigma-Aldrich (Canada), Gibco by life technologies (New Zealand), MP Biomedical (MP, USA), Rocky Mountain Biologicals (USA), and Thermo Fisher. The samples were thawed, aliquot and re-frozen and thawed once before being used and the remainder discarded. LEDGF (Lens Epithelium Derived Growth Factor) recombinant protein was supplied from MyBioSource (San Diego, USA). LEDGF recombinant protein was produced from Mus musculus (mouse) cells in culture and 0.1 mg of LEDGF was re-suspended in sterile PBS at concentration of before use as described. Insulin, human recombinant was supplied from Sigma-Aldrich (Canada). Insulin, human recombinant was dissolved in sterile PBS to a concentration of before use. All other salts and reagents were obtained from Sigma-Aldrich-Fluka (St Louis, MO) except where indicated.

### Protein Separation Over Quaternary Amine Resin

The proteins in 25 uL serum were diluted in 200 μL of 20 mM Tricine pH 8.8 binding buffer.

A new disposable preparative quaternary amine columns was created for each protein sample (Tucholska, M., et al., Human serum proteins fractionated by preparative partition chromatography prior to LC-ESI-MS/MS. Journal of proteome research, 2009. 8: p. 1143-1155). A 100 μL column of quaternary amine resin was struck in a 1.5 mL transfer pipette inside of a 15 mL tube (Marshall, J., et al., Human serum proteins preseparated by electrophoresis or chromatography followed by tandem mass spectrometry. J Proteome Res, 2004. 3(3): p. 36482). Quaternary amine resin in 50% slurry was packed by cutting off the bulb to act as a buffer reservoir and packing the tip with a glass wool frit (Tucholska, M., et al., Human serum proteins fractionated by preparative partition chromatography prior to LC-ESI-MS/MS. Journal of proteome research, 2009. 8: p. 1143-1155). The resin was equilibrated with binding buffer prior to introducing the sample by gravity. The resin was washed with 3 volumes of buffer prior to an increasing salt step-gradient. In order to avoid cross-contamination the preparative quaternary amine column was discarded after a single use. The samples were then digested with 1/100 wt/wt trypsin for 12 hours, reduced in 2 mM DTT for 30 minutes at 50° C. before digesting a second time with 1/100 wt/wt trypsin. The samples were quenched with 5% formic acid and dried.

### Endogenous Peptide Extraction

Serum samples (200 μL) were precipitated with 9 volumes of acetonitrile (90% ACN) (Tucholska, M., et al., Endogenous peptides from biophysical and biochemical fractionation of serum analyzed by matrix-assisted laser desorption/ionization and electrospray ionization hybrid quadrupole time-of-flight. Analytical biochemistry, 2007. 370: p. 228-245) in 2 mL sample tubes followed by stepwise extraction of the pellet. The acetonitrile/water extract was separated from the insoluble pellet at each step fraction with a centrifuge at 12,000 RCF for 5 minutes. The organic precipitate (pellet) that contains a much larger total amount of endogenous polypeptides was manually re-suspending in steps of increasing water content to yield 10 fractions from 90% ACN to 10% ACN, followed by water and then 5% formic acid (Dufresne, J., et al., A method for the extraction of the endogenous tryptic peptides (peptidome) from human EDTA plasma. Anal Biochem, 2018). The acetonitrile/water phase that contains peptides was collected, transferred to a fresh sample tube, dried under vacuum in a rotary lyophilizer, and stored at −80° C. for subsequent analysis.

### Preparative C18 Chromatography of Peptides

Preparative C18 separation provided the best results for peptide and phosphopeptide analysis in a “blind” comparison (Krokhin, O. V., W. Ens, and K. G. Standing, MALDI QqTOF MS combined with off-line HPLC for characterization of protein primary structure and post-translational modifications. J Biomol Tech, 2005. 16(4): p. 429-40). A new disposable C18 preparative “Zip Tip” column was used to collect each sub-fraction. Solid phase extraction with C18 for LC-ESI-MS/MS was performed as previously described. The C18 chromatography resin (Zip Tip) was wet with 65% acetonitrile before equilibration in water with 5% formic acid. The plasma extract was dissolved in 200 μL of 5% formic acid in water. The resin was washed with at least five volumes of the same binding buffer. The resin was eluted with >3 column volumes of 65% acetonitrile (2 μL) in 5% formic acid. In order to avoid cross-contamination the preparative C18 resin was discarded after a single use.

### Analytical LC-ESI-MS/MS

In order to entirely prevent any possibility of cross contamination between serum samples, a new disposable nano analytical HPLC column and nano emitter were fabricated for recording each set of quaternary amine (proteomics) and organic (peptidomic) extractions (n=3 fetal plus n=3 adult samples×2 fractionation methods=12 columns). Each fetal or adult serum sample resulted in two sample-fraction sets (10 peptide fractions and 16 protein fraction digests=26 fractions per sample) for a total of 156 LC-ESI-MS/MS experiments (2 treatments×3 replicates×26 fractions). The ion traps were cleaned and tested for sensitivity with angiotensin and glu-fibrinogen prior to recordings (Dufresne, J., et al., Re-evaluation of the rabbit myosin protein standard used to create the empirical statistical model for decoy library searching. Anal Biochem, 2018. 560: p. 39-49; Thavarajah, T., et al., Re-evaluation of the 18 non-human protein standards used to create the Empirical Statistical Model for Decoy Library Searching. Anal Biochem, 2020: p. 113680). Each disposable C18 analytical column was conditioned and quality controlled with a mixture of three non-human protein standards using a digest of Bovine Cytochrome C, Yeast alcohol dehydrogenase (ADH) and rabbit Glycogen Phosphorylase B to confirm the sensitivity and mass accuracy of the system (Bowden, P., et al., Quantitative statistical analysis of standard and human blood proteins from liquid chromatography, electrospray ionization, and tandem mass spectrometry. Journal of proteome research, 2012. 11: p. 2032-2047). The conditioned column was extensively washed in 50% acetonitrile before introducing the sample fraction set. The stepwise fractions were collected over C18 preparative micro columns, eluted in 24, of 65% ACN and 5% formic acid, diluted with 18 μL of 5% formic acid in water and immediately loaded manually into a 20 μL metal sample loop before injecting onto the analytical column via a Rhodynne injector. The peptide samples were analyzed over a discontinuous gradient at a flow rate of −10 uL per minute generated with an Agilent 1100 series capillary pump and split upstream of the injector during recording to about −200 nL per minute. The analytical HPLC separation was performed with a C18 Zorbax 5 micron 300 Angstrom (150 mm×0.15 mm) flitted capillary column. The acetonitrile profile was started at 5%, ramped to 12% after 5 minutes and then increased to 65% over −90 minutes, remained at 65% for 5 minutes, decreased to 50% for 15 minutes and then declined to a final proportion of 5% prior to injection of the next step fraction from the same adult or fetal serum sample. The nano HPLC effluent was analyzed by ESI ionization with detection by MS and fragmentation by MS/MS with a linear quadrupole ion trap (Schwartz, J. C., M. W. Senko, and J. E. Syka, A two-dimensional quadrupole ion trap mass spectrometer. J Am Soc Mass Spectrom, 2002. 13(6): p. 659-69). The device was set to collect the precursor for up to 200 milli seconds prior to MS/MS fragmentation with up to four MS/MS fragmentations per precursor ion.

### Correlation Analysis

In this study 1,554,347 precursor ions from fetal versus adult MS/MS spectra were recorded by nano LC-ESI-MS/MS. Correlation analysis of ion trap data was performed with X!TANDEM (Craig, R. and R. C. Beavis, TANDEM.•matching proteins with tandem mass spectra. Bioinformatics, 2004. 20(9): p. 1466-7) and SEQUEST (Yates, J. R., 3rd, et al., Method to correlate tandem mass spectra of modified peptides to amino acid sequences in the protein database. Anal Chem, 1995. 67(8): p. 1426-36) algorithms to match tandem mass spectra to peptide sequences from a library of 209,111 bovine proteins that differed by at least one amino acid from RIKEN, IMAGE, RefSeq, NCBI, Swiss Prot, TrEMBLE, ENSEMBL, UNIPROT and UNIPARC along with available Gene Symbols, all previous accession numbers, description fields and any other available annotation that was rendered non-redundant by protein sequence in SQL Server. Identified peptides with precursors greater than 10,000 (E4) arbitrary counts were accepted as fully tryptic peptides and/or tryptic phosphopeptides on separate servers for each algorithm and the results combined, and compared in SQL Server/R. The X!TANDEM default ion trap data settings of ±3 m/z from precursors peptides considered from 300 to 2000 m/z with a tolerance of 0.5 Da error in the fragments were used. The best fit peptide of the MS/MS spectra to fully tryptic (TRYP) and/or tryptic phosphopeptides (STYP) at charge states of +2 versus +3 were accepted with additional acetylation, or oxidation of methionine and with possible loss of water or ammonia.

### Data Analysis Transformation and Visualization

The linear quadrupole ion trap provided the precursor ion intensity values and the peptide fragment MS/MS spectra that were correlated to specific tryptic peptides (TRYP) or tryptic phosphopeptides (STYP) by the X! TANDEM and SEQUEST algorithms. The protein accession numbers, actual and estimated peptide masses, correlated peptide sequences, peptide and protein scores, resulting protein sequences and other associated data were captured and assembled together in an SQL Server relational database. The MS and MS/MS spectra together with the results of the X! TANDEM and SEQUEST algorithms were parsed into an SQL Server database and redundant fits of MS/MS spectra were filtered to the best hit before graphical and statistical analysis with the generic R data system. After correcting the observation frequency for the number of MS/MS spectra recorded in fetal versus adult experiments, the peptide to protein correlation counts for each gene symbol were compared for fetal versus adult serum using the Chi Square test using equation #1:

i) (Fetal−Adult)\hat{ }2/(Adult+1) EQN #1

The precursor intensity data for MS/MS spectra were logio transformed, tested for normality and analyzed across the adult versus fetal treatments, fractions, and replicates by means, standard errors and ANOVA (Bowden, P., et al., Quantitative statistical analysis of standard and human blood proteins from liquid chromatography, electrospray ionization, and tandem mass spectrometry. Journal of proteome research, 2012. 11: p. 2032-2047; Florentinus, A. K., et al., The Fc receptor-cytoskeleton complex from human neutrophils. Journal of proteomics, 2011. 75: p. 450-468).

### Cell Culture

All media, blood samples, supplement and reagents were sterilized prior to cell culture. Micropipettors, pipette tips, micro cover glasses, and cell culture flasks/plates were autoclaved in order to create an aseptic environment and no antibiotic were employed. A volume of 25 mL FBS was added to a 500 mL DMEM (5% serum). A 0.5 mL aliquot of frozen 3^{rd} passage of raw cells 264.7 from ATTC were diluted with 3 mL 5% FBS in DMEM in a cell culture flask and incubated in a humidified atmosphere of 5% CO2 at 37° C. until confluent. Media was changed after the first four hours since the raw cells contained DMSO when it was frozen down in the liquid nitrogen. Cells were passaged and scraped when they became 80% confluent. The effects of each serum on cell growth: three independent batches of FCS from different sources and three independent treatments of ABS from different sources assay were compared. Cells were seeded in 6-well plates with maximum 2 mL of 5% (v/v) FBS and 5% (v/v) ABS in DMEM respectively. The cells were cultured in the incubator at 37° C. and sampled (passaged) every 48 hours. The rate of cell growth was measured by looking at the confluence of the cells under a light microscope and the cell death and disintegration of large cells in ABS over time noted compare to cells in FCS that proliferated. For hormone experiments, confluent 5^{th} passage cells were plated at −30% in medium with 5% ABS and the amount of hormone indicated compared to 5% FCS. For cell size assays and imaging the media was removed and each cover slip with cells adhered to it was washed 3 times with 2 mL 1×PBS. 2 mL of 2% Para-formaldehyde was added to each well for cell fixation for no more than 2 hours. Para-formaldehyde was removed and the cells were washed 3 times with 2 mL 1×PBS before staining with Rhodamine phalloidin and imaging under a Zeiss 520 Metal laser confocal microscope.

### Results

### Cell Morphology

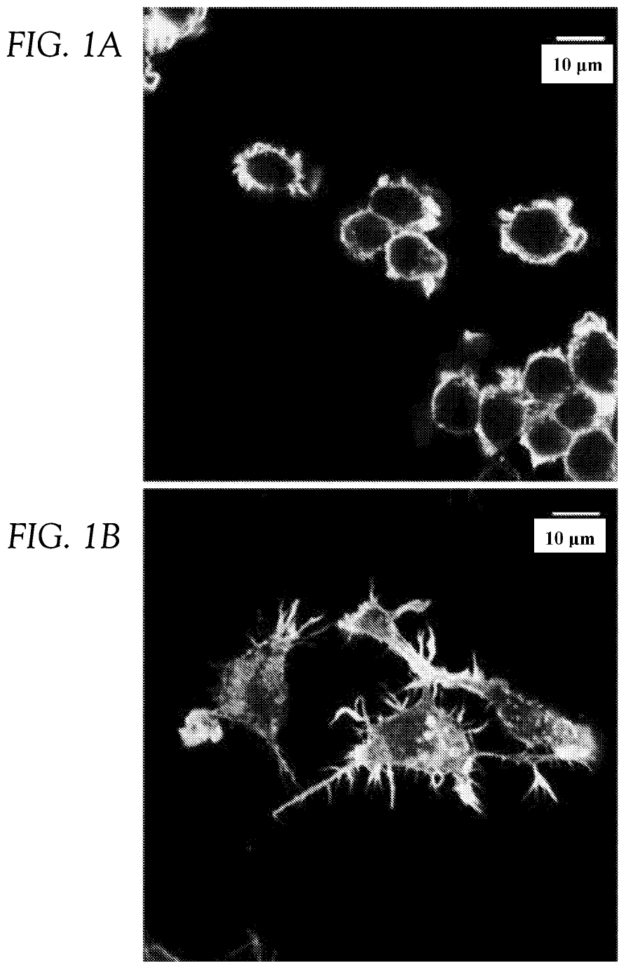

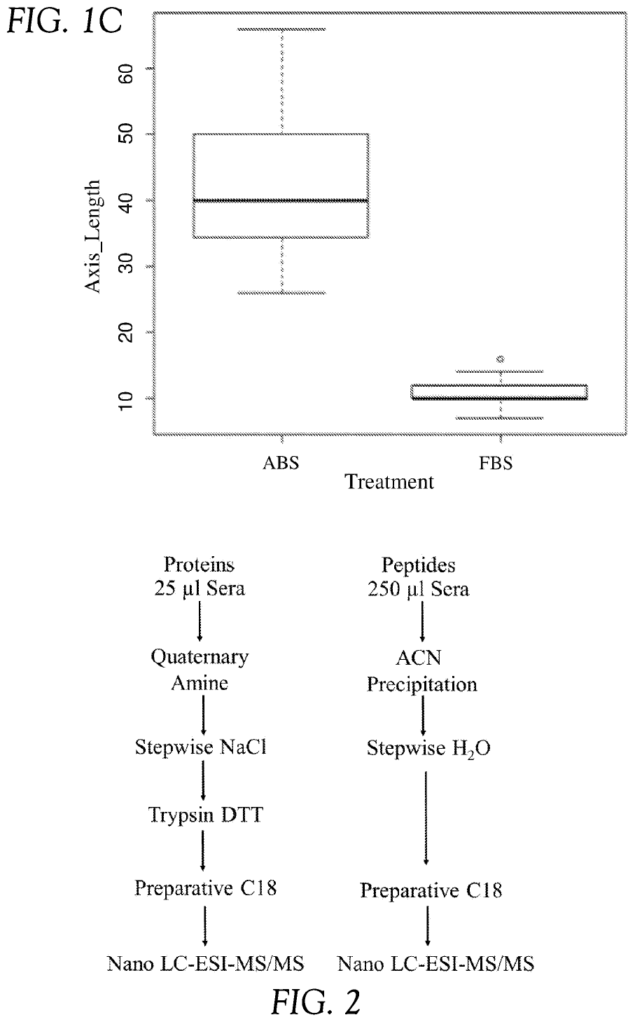

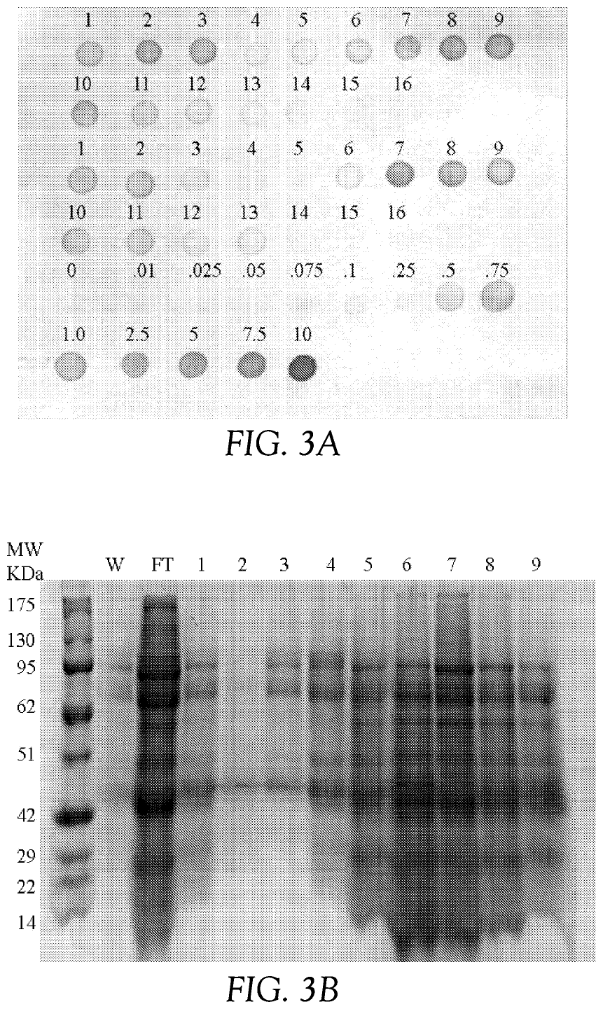

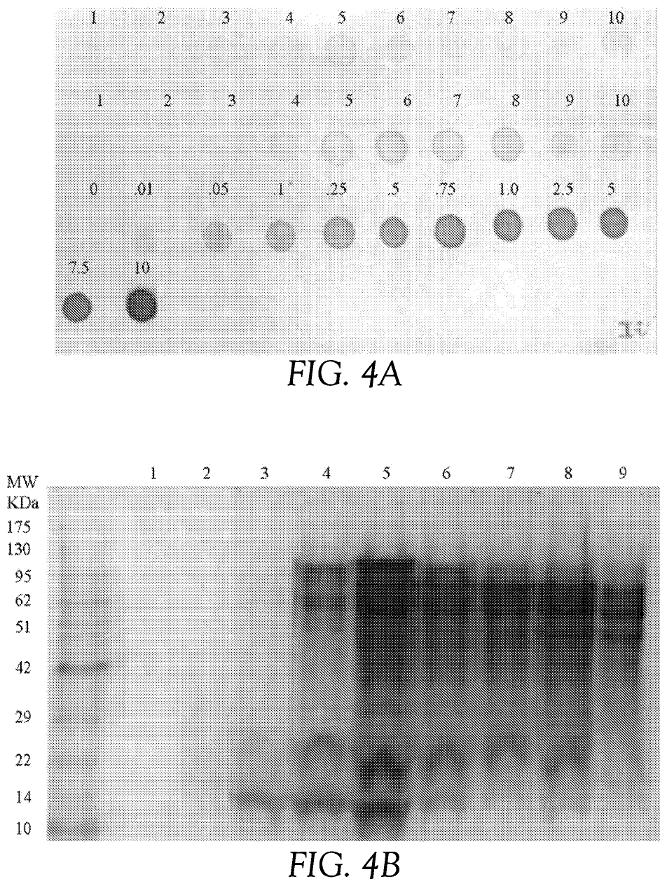

The cells grown in fetal serum divided rapidly, as reflected by small cell length, and formed foci organized into a globe of cells that extended vertically upwards piled onto each other. In contrast, cells cultured in adult serum formed a monolayer of elongated rhomboids with dendritic extensions that divided slowly and eventually died. Staining with rhodamine phalliodin showed that fetal serum resulted in rounded symmetrical cells that average 10 microns across while adult serum produced elongated cells that were about 40 microns in length (FIG. 1).

### Stepwise Fractionation of Peptides and Proteins

The proteins of fetal calf serum compared to adult serum were separated over ceramic quaternary amine resin and the fractions tested for protein content by the Dumbroff dot blotting method prior to resolving the proteins by tricine SDS-PAGE that showed selectivity over the course of the salt step gradient (FIGS. 2&3). The organic precipitate of endogenous peptides from fetal calf serum compared to adult serum were separated by a water step gradient and differential centrifugation, tested for protein content by the Dumbroff method (Ghosh, S., et al., Use of a scanning densitometer or an ELISA plate reader for measurement of nanogram amounts of protein in crude extracts from biological tissues. Anal Biochem, 1988. 169(2): p. 227-33) before resolving the polypeptides on tricine SDS-PAGE that showed selectivity for low molecular mass polypeptides (FIGS. 2&4). The optimal organic solvent composition for endogenous peptide extraction was from 40 to 60% acetonitrile while the optimal salt concentration for intact protein elution from quaternary amine resin was 100 to 175 mM NaCl (Table I).

TABLE I
Protein separation over the salt fractions versus endogenous
peptide separation over organic water on the proteins
and tryptic peptides identified (See FIG. 2).

| Peptides | | | | |
|---|---|---|---|---|
| Fraction | Concentration | Redundant | Distinct | Distinct |
| Number | (%) | Protein | Protein | Peptide |
| 1 | 0% AcN | 223397 | 44397 | 48853 |
| 2 | 5% AcN | 249899 | 46250 | 55977 |
| 3 | 10% AcN | 250107 | 45728 | 53953 |
| 4 | 20% AcN | 236723 | 44892 | 49899 |
| 5 | 30% AcN | 249846 | 46750 | 58077 |
| 6 | 40% AcN | 263797 | 48017 | 63652 |
| 7 | 50% AcN | 271287 | 47568 | 62042 |
| 8 | 60% AcN | 266640 | 47248 | 62155 |
| 9 | 70% AcN | 278624 | 46975 | 59369 |
| 10 | 90% AcN | 274823 | 46150 | 54845 |
| | | | 463975 | 568822 |
| Proteins | | | | |
| Fraction | Concentration | Redundant | Distinct | Distinct |
| Number | (mM) | Protein | Protein | Peptide |
| 1 | 0 | 430748 | 51459 | 87107 |
| 2 | 50 | 393872 | 50332 | 79094 |
| 3 | 100 | 517203 | 52308 | 91084 |
| 4 | 150 | 478902 | 52205 | 93276 |
| 5 | 175 | 530335 | 53440 | 109606 |
| 6 | 200 | 379348 | 50820 | 83834 |
| 7 | 225 | 324976 | 50150 | 78085 |
| 8 | 250 | 278455 | 48049 | 64995 |
| 9 | 300 | 382052 | 50746 | 81509 |
| 10 | 350 | 351982 | 49386 | 73052 |
| 11 | 400 | 326096 | 48878 | 69684 |
| 12 | 450 | 261822 | 46261 | 57871 |
| 13 | 500 | 404293 | 50257 | 79141 |
| 14 | 600 | 322479 | 48271 | 67073 |
| 15 | BdDigest | 211181 | 43258 | 46105 |
| 16 | QF | 399748 | 50099 | 77835 |
| | | | 795919 | 1239351 |

### Normality and Variation Across Treatments and Replicates

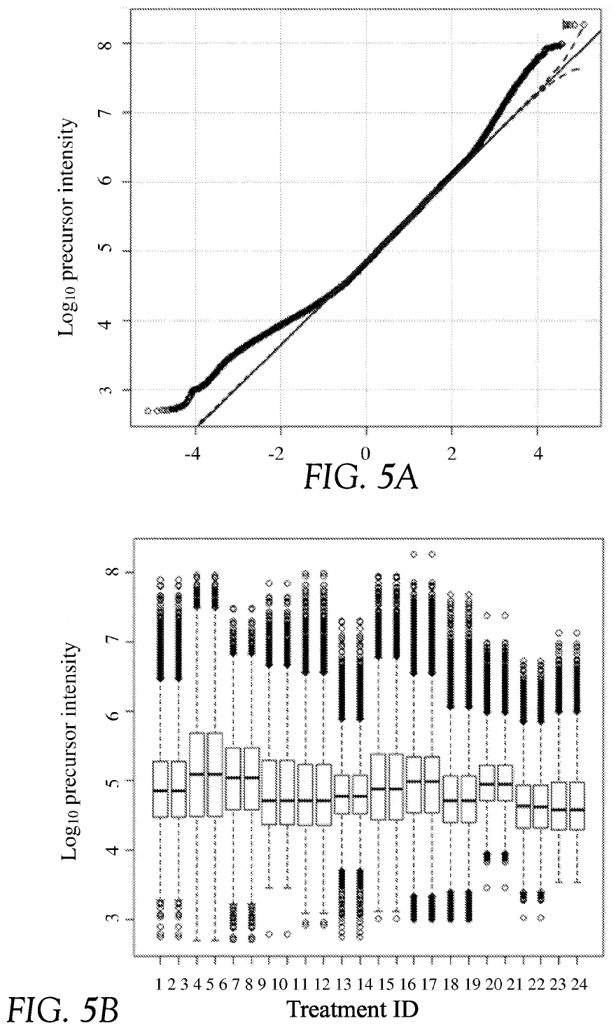

The logio precursor intensity values from all treatments and replicates together approached a linear and Gaussian distribution (FIG. 5A). The average results of the 6 experiments (2 treatments×3 replicates) were comparable in terms of the intensity of the precursor peptides obtained with and without the consideration of phosphorylation (FIG. 5B).

### SQL Server Filtering

The pool of tryptic peptides from proteins and endogenous tryptic (TRYP) peptides and/or tryptic phosphopeptides (STYP) were randomly and independently sampled without replacement by liquid chromatography, nano electrospray ionization and tandem mass spectrometry (LC-ESI-MS/MS) (Dufresne, J., et al., Random and independent sampling of endogenous tryptic peptides from normal human EDTA plasma by liquid chromatography micro electrospray ionization and tandem mass spectrometry. Clin Proteomics, 2017. 14: p. 41) from fetal bovine serum (FBS) versus adult bovine serum (ABS). The raw correlations from precursors >E4 intensity counts were filtered to retain only the best fit by charge state and peptide sequence in SQL Server to entirely avoid re-use of the same MS/MS spectra. The LC-ESI-MS/MS of serum recorded 1,553,347 MS/MS spectra resulting in 61,152 tryptic correlations (3.9%) by the X!TANDEM (Table II).

TABLE II
The filtering of the MS/MS spectra and the resulting correlations to peptides to ensure that
only the best hit was accepted. The total number of MS/MS spectra of greater than E4 counts
collected in this study was 526,870 MS/MS spectra from organic extraction and 1,027,477 from
quaternary amine fractionation of proteins followed by digestion resulting in a total of
1,554,347 MS/MS spectra. Treatments: TRYP, fully tryptic with <3 missed cleavages; TRYP
STYP, fully tryptic with optional phosphorylation of serine, threonine or tyrosine. The Bovine
protein library contained 209,111 proteins that differed by at least 1 amino acid.

| | Total | Redundant | Distinct | | Distinct | Distinct |
|---|---|---|---|---|---|---|
| Redundant | Spectra | MSMS | MSMS | Protein | Protein | Peptide |
| MS/MS | Count | Spectra | Spectra | Identifications | Identifications | Identifications |
| TRYP | 2616468 | 36560334 | 2353004 | 36560334 | 204332 | 2777338 |
| TRYP | 2616468 | 36297856 | 2351922 | 36297856 | 201220 | 2598348 |
| STYP | | | | | | |

| | total | Redundant | Distinct | | Distinct | Distinct |
|---|---|---|---|---|---|---|
| Best Fit | spectra | MSMS | MSMS | Protein | Protein | Peptide |
| Spectra | count | Spectra | Spectra | Identifications | Identification | Identifications |
| TRYP | 2616468 | 7782701 | 2353004 | 7782701 | 179653 | 1063402 |
| TRYP | 2616468 | 7730807 | 2351922 | 7730807 | 176176 | 1040077 |
| STYP | | | | | | |
| TOTAL | 5232936 | | | | | |

| | | Redundant | Distinct | | Distinct | Distinct |
|---|---|---|---|---|---|---|
| Best Fit | | MSMS | MSMS | Protein | Protein | Peptide |
| Algorithm | Peptide | Spectra | Spectra | Identifications | Identifications | Identifications |
| X!TANDEM | TRYP | 383535 | 127250 | 383535 | 57308 | 61152 |
| X!TANDEM | TRYP | 342083 | 116827 | 342083 | 44146 | 57435 |
| | STYP | | | | | |
| SEQUE ST | TRYP | 7399166 | 2344666 | 7399166 | 178895 | 1021440 |
| SEQUE ST | TRYP | 7388724 | 2345002 | 7388724 | 176133 | 998966 |
| | STYP | | | | | |

### Peptide and Protein Identification

There was little agreement at the level of peptides from peptidomics versus proteomics with 348,543 peptides observed only from exogenous digestion of proteins (proteomics) and 142,197 peptides only observed from endogenous peptides (peptidomics). There was −99.3% agreement on the proteins independently identified by peptidomics versus proteomics. The 225,097 distinct endogenous peptide sequences from peptidomics showed 23% overlap with the 431,443 distinct peptide sequences from proteomics but showed near perfect agreement on the set of proteins that were identified. Less than one third of all bovine proteins were identified with at least 5 or more peptides. In contrast, the majority of the known 183,426 bovine protein accessions were never observed. That alone is sufficient evidence to demonstrate the veracity of LC-ESI-MS/MS of blood peptides with a simple ion trap (Bowden, P., et al., Meta sequence analysis of human blood peptides and their parent proteins. Journal of proteomics, 2010. 73: p. 1163-1175). However even though the two sets of peptide sequences observed differed dramatically, they were derived from the same set of parent proteins: organic extraction of endogenous peptides (peptidomics) identified a set of 58,200 protein accessions that showed near perfect overlap and agreement with the set of 59,799 accessions from protein separation and tryptic digestion (proteomics) (Table

TABLE III
Comparison of organic extraction of peptides versus separation
of proteins over quaternary amine chromatography. The redundant
and distinct proteins and peptides and the overlap between
treatments were computed in SQL Server (see FIG. 2).

| Total | Organic | Salt | Common | Found | Found |
|---|---|---|---|---|---|
| Distinct | Distinct | Distinct | Distinct | Only in | Only in |
| Proteins | Proteins | Proteins | Proteins | Organic | Salt |
| 60233 | 58200 | 59799 | 57766 | 434 | 2033 |
| Total | Organic | Salt | Common | Found | Found |
| Distinct | Distinct | Distinct | Distinct | only in | Only in |
| peptides | peptides | peptides | Peptides | Organic | Salt |
| 573640 | 225097 | 431443 | 82900 | 142197 | 348543 |

### The Protein, Accessions and Gene Symbols of Bovine Serum

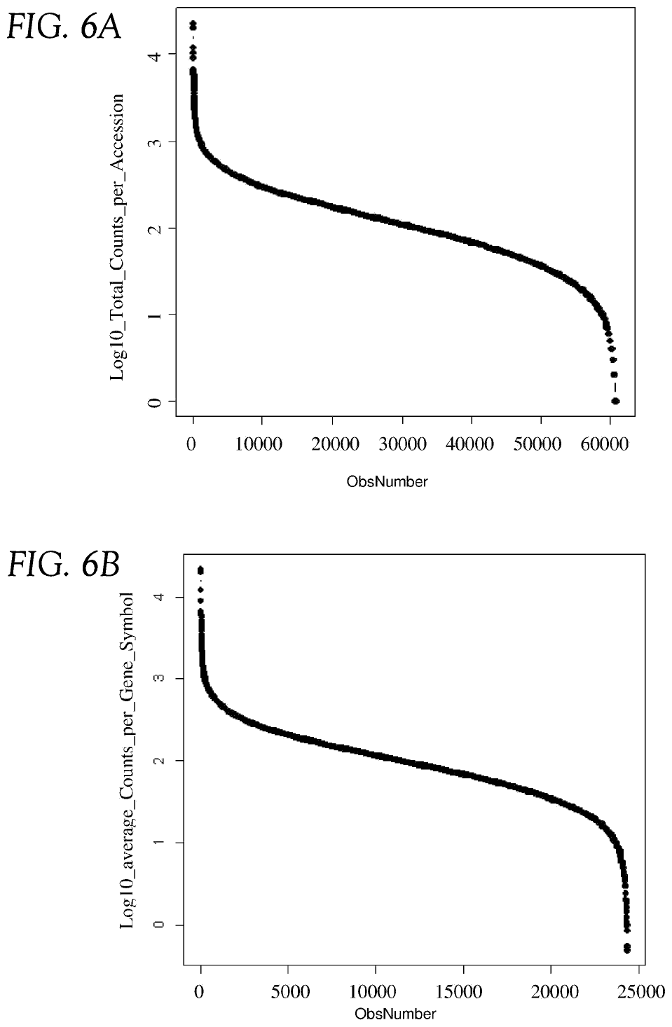

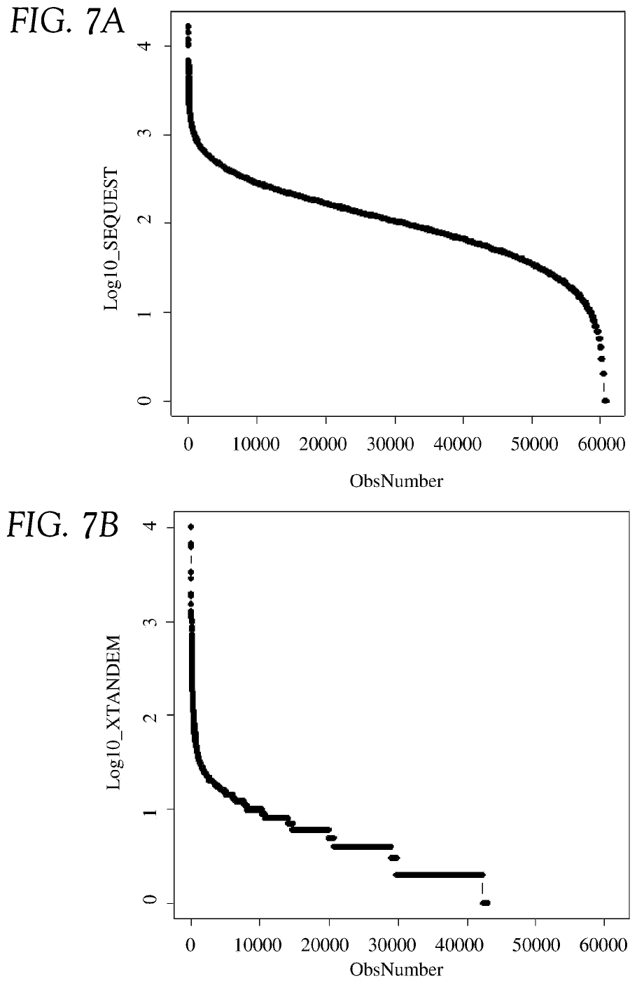

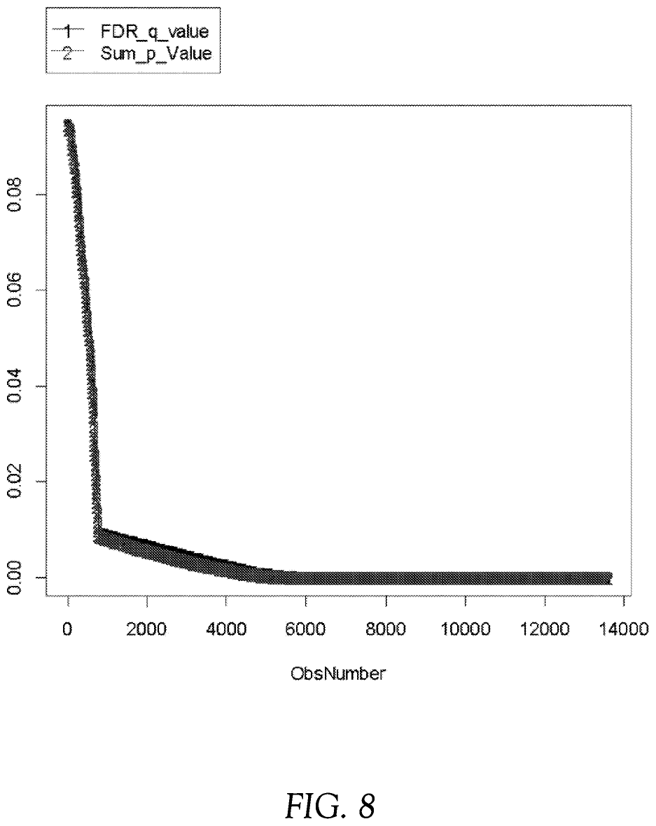

X!TANDEM identified some 12,000 proteins gene symbols with multiple peptides (FIG. 7) while the SEQUEST algorithm provided the observation frequency count. The peptide that was the best fit of MS/MS spectra after considering the fit of charge state and sequence were then analyzed by the generic R statistical system showed that −59,000 protein accessions and more than 20,000 protein gene symbols were detected by the sum of the X! TANDEM and SEQUEST results with at least 5 peptides (FIG. 6) that should confer near certain identification. X!TANDEM permitted computation of cumulative p-values and FDR corrected q-values for each gene symbol by the method of Benjamini and Hochberg (Zhu P-H, K. K., Jankowski A, Marshall J, Comparison of published human serum/plasma data. Molecular and Cellular Proteomics, 2004. 3(0.1): p. 5235) that showed some 12,000 protein gene symbols with a q-value of 0.01 or less (FIG. 8).

### Fetal Proteins

Proteins that showed highly significant increases in observation frequency in the fetal serum (x2 value >50) included alpha fetoprotein that is known to be expressed in the fetal liver and shows about 39% homology with BSA and 20% homology to vitamin D binding protein by BLAST (Table IV). Common serum proteins specifically increased in fetal serum include alpha-2-HS-glycoprotein precursor, serpin peptidase inhibitor Glade A, fetuin B, gamma globin, inter-alpha-trypsin inhibitor heavy chain H3, collagen and calcium-binding EGF domain-protein, pyruvate carboxylase, adenylate kinase, hemoglobin subunit beta, kallikrein K, thrombospondin 4, and ATP Synthase Membrane Subunit (ATP5MD). The fetal serum was enriched in Insulin-like Growth Factor II as expected (Yao, T. and Y. Asayama, Animal-cell culture media: History, characteristics, and current issues. Reprod Med Biol, 2017. 16(2): p. 99-117).

TABLE IV
Fetal serum specific proteins detected by fully tryptic peptides and fully tryptic
serine, threonine or tyrosine phosphopeptides from X!TANDEM and/or SEQUEST that
show a corrected observation frequency difference (Delta) and Chi Square (x2) value
of >50 for both tryptic (TRYP) and phosphotyptic (STYP) peptides.

| Gene_Symbol | Count | TANDEM | SEQUEST | TRYP_X2 | TRYP_Delta | STYP_X2 | STYP_Delta |
|---|---|---|---|---|---|---|---|
| AHSG | 13062 | 7364 | 5698 | 82229 | 6367 | 35346 | 2769 |
| SERPINA1 | 6313 | 3602 | 2711 | 39176 | 3008 | 21045 | 1429 |
| FETUB | 1256 | 575 | 681 | 29189 | 704 | 5503 | 235 |
| ITIH3 | 1663 | 757 | 907 | 8339 | 729 | 1733 | 303 |
| AFP | 2472 | 1222 | 1250 | 3512 | 903 | 948 | 351 |
| MEPCE | 4081 | 6 | 4075 | 2641 | 1352 | 14 | 22 |
| LOC 104974567 | 6646 | 4 | 6642 | 2475 | 1125 | 2295 | 1098 |
| PDCD4 | 6731 | 2 | 6729 | 2459 | 1125 | 2230 | 1104 |
| DGAT1 | 10513 | 6 | 10507 | 1894 | 1292 | 2494 | 1749 |
| RRAGB | 432 | 6 | 426 | 1776 | 146 | 1921 | 131 |
| SH2B1 | 5892 | 11 | 5881 | 1763 | 866 | 2185 | 1121 |
| SPRY4 | 2836 | 14 | 2822 | 1626 | 551 | 1421 | 604 |
| KIRREL3 | 5220 | 0 | 5220 | 1397 | 739 | 1919 | 975 |
| FBLL1 | 3615 | 125 | 3490 | 1187 | 546 | 862 | 505 |
| CCDC9 | 6014 | 20 | 5994 | 1180 | 683 | 2005 | 1153 |
| NFATC2IP | 5805 | 18 | 5787 | 977 | 594 | 1654 | 1077 |
| SFPQ | 5838 | 31 | 5807 | 906 | 568 | 1643 | 1089 |
| HMX1 | 5729 | 19 | 5710 | 904 | 566 | 1634 | 1070 |
| L2HGDH | 2991 | 0 | 2991 | 899 | 445 | 825 | 1499 |
| GNPTG | 575 | 16 | 559 | 865 | 140 | 405 | 136 |
| BRWD3 | 6084 | 10 | 6074 | 846 | 575 | 1583 | 1081 |
| FGA | 461 | 160 | 301 | 778 | 146 | 141 | 57 |
| PLA2G2A | 4547 | 4 | 4543 | 768 | 556 | 1160 | 797 |
| LOC 101902853 | 262 | 0 | 262 | 762 | 78 | 367 | 74 |
| LOC 100125947 | 4549 | 4 | 4545 | 762 | 555 | 1165 | 798 |
| ADGRV1 | 4428 | 26 | 4402 | 757 | 512 | 1351 | 830 |
| LOC788663 | 162 | 2 | 160 | 720 | 71 | 26 | 14 |
| CTR9 | 6140 | 27 | 6113 | 687 | 546 | 1472 | 1056 |
| MORF4L1 | 3149 | 2 | 3147 | 637 | 434 | 613 | 458 |
| NXNL2 | 2112 | 4 | 2108 | 606 | 313 | 591 | 354 |
| H2AFX | 2581 | 6 | 2575 | 603 | 333 | 718 | 434 |
| LOC100138633 | 596 | 8 | 588 | 598 | 129 | 338 | 134 |
| RBMX | 3505 | 28 | 3478 | 578 | 351 | 947 | 632 |
| ETFA | 1652 | 2 | 1650 | 558 | 190 | 1323 | 442 |
| GAR1 | 3450 | 24 | 3426 | 478 | 313 | 818 | 596 |
| ZRSR2 | 1225 | 8 | 1217 | 475 | 317 | 20 | 28 |
| PPP1R12B | 2121 | 10 | 2111 | 469 | 247 | 763 | 440 |
| EME2 | 1947 | 4 | 1943 | 431 | 247 | 616 | 383 |
| NFKBIL1 | 1599 | 13 | 1586 | 419 | 231 | 408 | 264 |
| SUSD4 | 122 | 0 | 122 | 384 | 36 | 205 | 25 |
| LUM | 396 | 168 | 228 | 369 | 121 | 127 | 56 |
| BDKRB1 | 1477 | 2 | 1475 | 368 | 183 | 446 | 270 |
| IL2RA | 337 | 1 | 336 | 350 | 110 | 25 | 17 |
| LOC101902766 | 441 | 0 | 441 | 349 | 120 | 440 | 81 |
| AKT1 | 2310 | 8 | 2302 | 347 | 269 | 365 | 307 |
| MEETTL25 | 2270 | 4 | 2266 | 345 | 267 | 300 | 286 |
| PRDM11 | 463 | 216 | 247 | 335 | 89 | 115 | 67 |
| BAG4 | 235 | 0 | 235 | 329 | 44 | 204 | 59 |
| FBLN1 | 554 | 86 | 468 | 319 | 112 | 276 | 105 |
| FABP1 | 56 | 12 | 44 | 318 | 18 | 43 | 11 |
| LOC787241 | 235 | 0 | 235 | 316 | 56 | 211 | 58 |
| ITIH2 | 4294 | 2200 | 2095 | 312 | 537 | 74 | 188 |
| CCDC124 | 1475 | 0 | 1475 | 304 | 185 | 465 | 264 |
| MCAT | 375 | 203 | 172 | 293 | 80 | 126 | 71 |
| IK | 586 | 218 | 368 | 290 | 138 | 45 | 46 |
| FLJ10922 | 604 | 4 | 600 | 290 | 95 | 257 | 120 |
| KIAA2012 | 1116 | 14 | 1102 | 287 | 158 | 53 | 78 |
| TMEM143 | 611 | 4 | 607 | 286 | 95 | 241 | 119 |
| KEAP1 | 1264 | 3 | 1261 | 274 | 158 | 491 | 238 |
| TTLL10 | 1124 | 5 | 1119 | 270 | 155 | 243 | 168 |
| MR VI1 | 1436 | 9 | 1426 | 263 | 164 | 477 | 246 |
| ACOT6 | 223 | 123 | 100 | 257 | 54 | 113 | 43 |
| LEO1 | 3222 | 2 | 3220 | 256 | 206 | 308 | 409 |
| TMEM114 | 827 | 8 | 819 | 255 | 84 | 1007 | 275 |
| LGMN | 439 | 8 | 431 | 246 | 115 | 45 | 31 |
| AHDC1 | 5051 | 43 | 5008 | 240 | 214 | 1662 | 1149 |
| AMBP | 1045 | 403 | 642 | 238 | 233 | 41 | 52 |
| FUT2 | 218 | 0 | 218 | 237 | 45 | 312 | 53 |

### AKT HRAS Cellular Proteins in Plasma

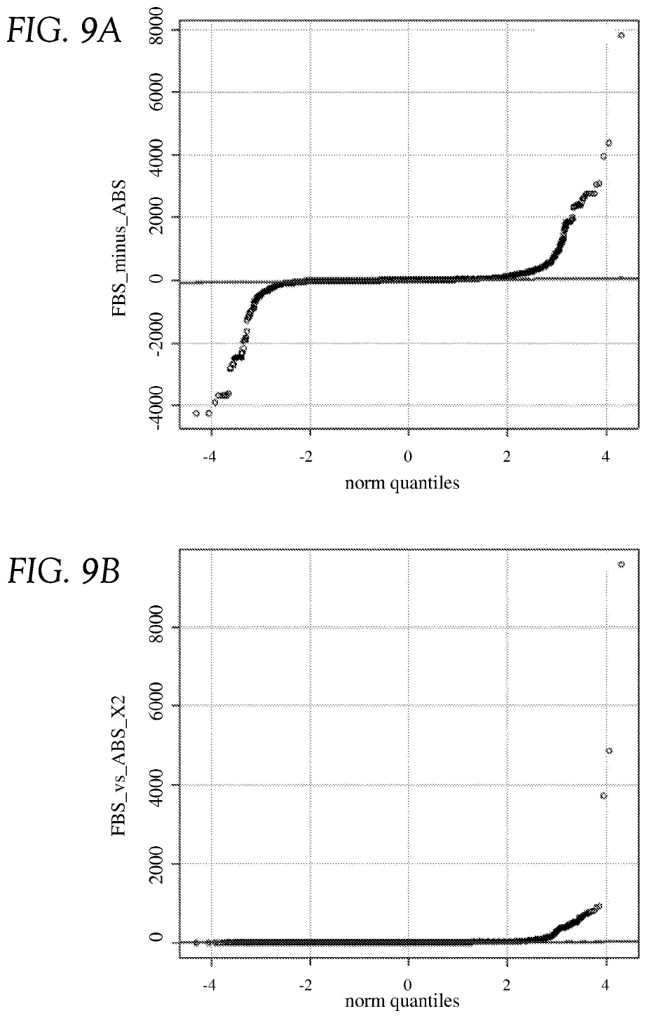

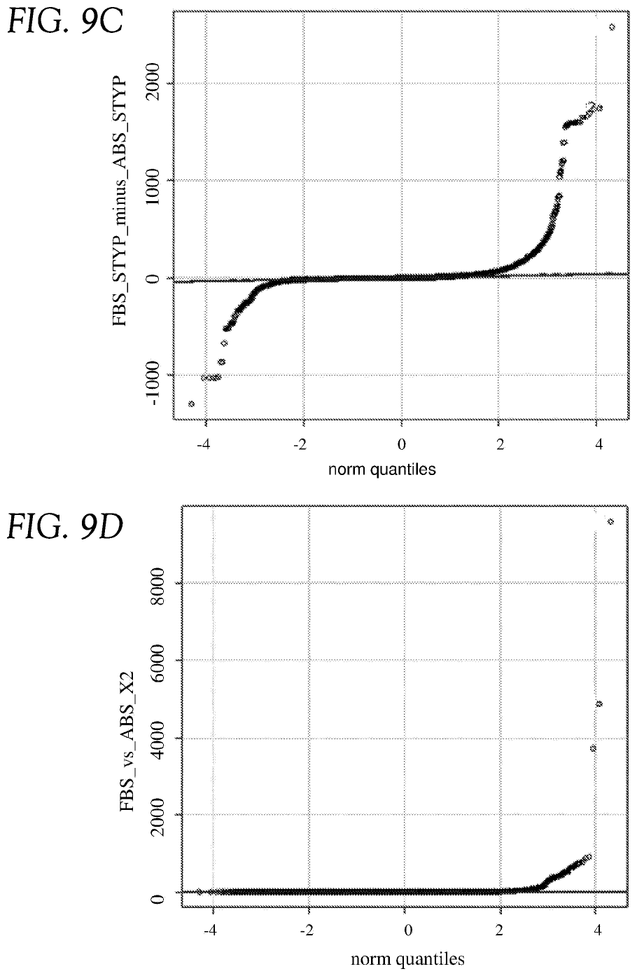

The Chi Square analysis showed some proteins with x2 values that were apparently far too large (x2≥60, p<0.0001, d.f. 1) to be resulted from random sampling error (FIG. 9). Chi Square analysis also identified many apparent cellular proteins such as ligands, receptors, signaling proteins, g-protein related, receptor enzymes like kinases, phosphatases, g-proteins, cyclases, phosphodiesterases, and proteins with interaction domains like SH2 protein as well as nucleic acid binding proteins including zinc fingers, histones, bromodomains, homeobox proteins and transcription factors that might contribute to cellular transformation (Marshall, J., et al., Creation of a federated database of blood proteins: a powerful new tool for finding and characterizing biomarkers in serum. Clin Proteomics, 2014. 11(1): p. 3), that were all apparently increased in fetal serum. The full list of proteins that were identified by X! TANDEM that showed greater frequency in fetal serum Chi Square (x2) values of >9 are shown in Table V. Additional example factors that were observed included nuclear receptor corepressor 2 isoform X2, Atrial natriuretic peptide receptor 2, scavenger receptor class A member 3, ALK tyrosine kinase receptor and many others.

TABLE V
The growth factors, cytokines, chemokines, interleukins and tumor necrosis factor that differ between
fetal versus adult serum by Chi Square analysis of the tryptic (TRYP) and or serine, threonine
or tyrosine phosphorylated tryptic peptides (STYP) identified by SEQUEST or X!TANDEM where
X2 of fully tryptic (TRYP) or phospho tryptic (STYP) peptides are greater than 4 (p < 0.05).

| Gene_Symbol | Count | X!TANDEM | SEQUEST | TRYP_Delta | TRYP_X2 | Delta_STYP | X2_STYP |
|---|---|---|---|---|---|---|---|
| CCL20 | 11 | 0 | 11 | 2 | 5 | 0 | 0 |
| CCL21 | 47 | 2 | 45 | 5 | 6 | 0 | 0 |
| CGRRF1 | 28 | 0 | 28 | 6 | 17 | 0 | 0 |
| CRLF1 | 146 | 37 | 109 | 11 | 10 | 9 | 4 |
| CXCR1 | 103 | 0 | 103 | 15 | 41 | 2 | 1 |
| CXCR2 | 51 | 0 | 51 | 6 | 11 | 1 | 0 |
| DOCK11 | 241 | 7 | 234 | 9 | 5 | −4 | 1 |
| EGR2 | 44 | 0 | 44 | 7 | 9 | −3 | 1 |
| EGR4 | 148 | 4 | 144 | 2 | 1 | 29 | 34 |
| FGF14 | 73 | 0 | 73 | −9 | 6 | −15 | 9 |
| FGF4 | 33 | 0 | 33 | −5 | 2 | 1 | 5 |
| FGF7 | 60 | 1 | 59 | −8 | 4 | −1 | 0 |
| Fgf8 | 60 | 0 | 60 | −12 | 8 | −3 | 1 |
| GDF11 | 690 | 6 | 684 | −48 | 18 | −47 | 15 |
| GDF11 | 690 | 6 | 684 | −48 | 18 | −47 | 15 |
| GDF5 | 318 | 8 | 310 | −9 | 2 | −47 | 24 |
| GDF6 | 140 | 2 | 138 | 7 | 4 | 13 | 9 |
| GPRIN1 | 668 | 54 | 614 | 44 | 20 | 2 | 0 |
| GPRIN2 | 54 | 4 | 50 | −2 | 1 | 5 | 6 |
| IGF2 | 172 | 6 | 166 | 38 | 57 | 8 | 5 |
| IGFBP2 | 45 | 0 | 45 | 2 | 4 | 6 | 17 |
| IGFBP4 | 199 | 7 | 192 | 26 | 27 | −4 | 1 |
| ILlORB | 80 | 1 | 79 | 13 | 18 | 6 | 9 |
| IL17C | 87 | 0 | 87 | −5 | 2 | 14 | 12 |
| IL17RC | 98 | 1 | 97 | 6 | 7 | 4 | 4 |
| IL18RAP | 158 | 6 | 152 | 13 | 15 | −2 | 0 |
| ILIA | 28 | 2 | 26 | 0 | 0 | 3 | 4 |
| IL1B | 82 | 2 | 80 | −2 | 0 | −8 | 5 |
| IL22RA2 | 59 | 2 | 57 | 7 | 6 | −1 | 0 |
| IL2RA | 113 | 0 | 113 | 36 | 114 | 6 | 8 |
| IL2RA | 113 | 0 | 113 | 36 | 114 | 6 | 8 |
| IL36A | 23 | 2 | 21 | 3 | 4 | −1 | 0 |
| L6 | 49 | 0 | 49 | −5 | 3 | 8 | 8 |
| IRAK1BP1 | 28 | 1 | 27 | 2 | 2 | 3 | 5 |
| Irak4 | 269 | 2 | 267 | 22 | 19 | 16 | 6 |
| IRAK4 | 130 | 1 | 129 | 8 | 8 | 6 | 2 |
| Irak4 | 269 | 2 | 267 | 22 | 19 | 16 | 6 |
| LOC 100848143 | 68 | 0 | 68 | 1 | 0 | 10 | 9 |
| LOC510185 | 58 | 2 | 56 | 10 | 19 | 1 | 0 |
| LOC784541 | 128 | 1 | 127 | 33 | 77 | 4 | 2 |
| LTBP4 | 184 | 4 | 180 | 8 | 7 | −2 | 0 |
| MEGF11 | 108 | 1 | 107 | 12 | 7 | 1 | 0 |
| NFIL3 | 134 | 0 | 134 | 10 | 8 | −1 | 0 |
| OGFR | 383 | 4 | 379 | −22 | 4 | −15 | 5 |
| OGFR | 383 | 4 | 379 | −22 | 4 | −15 | 5 |
| OSGIN2 | 93 | 1 | 92 | 6 | 5 | 10 | 8 |
| PDGFC | 76 | 5 | 71 | 5 | 4 | 1 | 0 |
| PDGFD | 59 | 0 | 59 | 6 | 5 | 2 | 1 |
| PDGFRL | 109 | 2 | 107 | −6 | 2 | 12 | 13 |
| PRP6 | 51 | 4 | 47 | 5 | 4 | −5 | 3 |
| RERG | 59 | 1 | 59 | −2 | 1 | −13 | 8 |
| SOCS5 | 133 | 2 | 131 | −11 | 5 | 8 | 16 |
| TGFB2 | 113 | 0 | 113 | 12 | 16 | −7 | 3 |
| TMEM219 | 16 | 0 | 16 | −1 | 0 | 3 | 26 |
| TNF | 45 | 3 | 42 | 8 | 9 | 2 | 1 |
| TNFa | 59 | 5 | 54 | 12 | 11 | 2 | 1 |
| TNFAIP8L1 | 45 | 2 | 43 | 5 | 12 | −2 | 0 |
| TNFAIP8L2 | 79 | 2 | 77 | 11 | 29 | 13 | 20 |
| TNF-alpha | 24 | 5 | 19 | 1 | 0 | 4 | 7 |
| TNFR2 | 9 | 0 | 9 | 4 | 16 | 0 | 0 |
| TNFRSF13 B | 91 | 1 | 90 | −6 | 2 | −19 | 13 |
| TNFRSF1B | 83 | 0 | 83 | −9 | 6 | −20 | 13 |
| TNFRSF25 | 99 | 2 | 98 | 18 | 34 | 0 | 0 |
| TNFSF12 | 52 | 2 | 49 | 6 | 5 | 1 | 2 |
| TNFSF18 | 27 | 0 | 27 | 1 | 1 | −5 | 4 |
| TNFSF4 | 23 | 0 | 23 | 4 | 4 | −2 | 1 |
| TRAF1 | 141 | 0 | 141 | −3 | 1 | 24 | 54 |
| USMGS | 242 | 0 | 242 | 65 | 108 | 4 | 15 |

### Insulin vs LEDGF

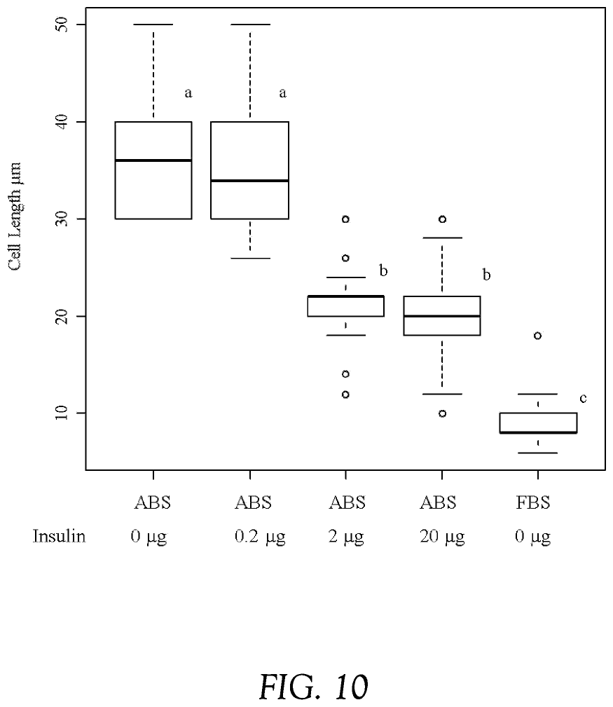

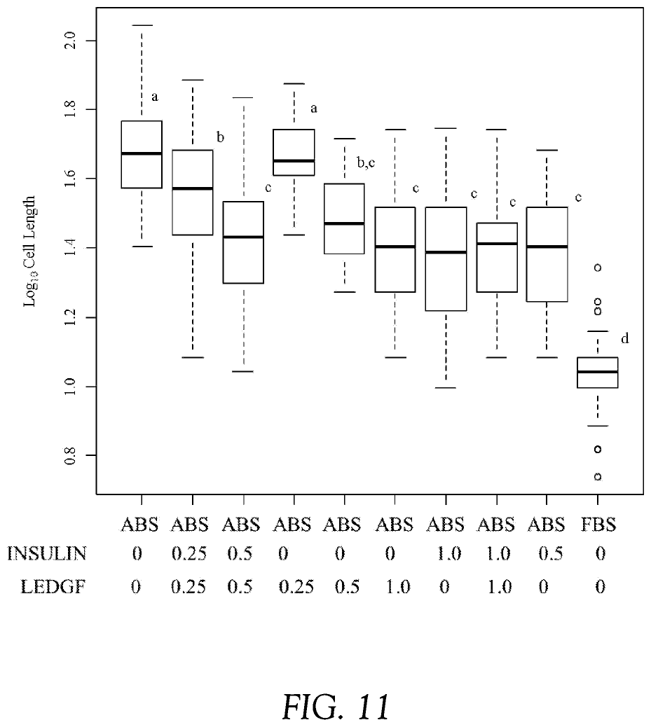

From the proteins that were increased in fetal bovine serum and showed significant x2 values and the literature, we selected insulin and LEDGF to test in a cell growth assay. In agreement with previous results (Lieberman, I. and P. Ove, Growth factors for mammalian cells in culture. J Biol Chem, 1959. 234: p. 2754-8), the addition of insulin to the adult bovine serum restored some of the rounded, non-differentiated phenotype associated with rapid cellular proliferation but was not as effective as fetal bovine serum. The IC50 of insulin was approximately 1 μg per mL (FIG. 10). Similarly, the addition of Lens Epitheliam-Derived Growth Factor (LEDGF) alone, or together with insulin, partially restored the rounded phenotype but was not as effective as complete fetal serum (FIG. 11). The analysis revealed growth factors specific to fetal serum such as Lens Epidermal Derived Growth Factor (LEDGF) apparently regulates cells size and development. The results of the add-back experiment were also consistent with the fidelity and sensitivity of LC-ESI-MS/MS of serum to detect low abundance growth factors specific to fetal serum.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

1. A method for stimulating growth of a cell comprising contacting the cell with a composition comprising an isolated lens epidermal dermal growth factor (LEDGF).

2. (canceled)

3. The method of claim 1, wherein the composition further comprises a hormone.

4. The method of claim 3, wherein the hormone is a growth related hormone.

5. The method of claim 3, wherein the hormone is selected from the group consisting of insulin, insulin like growth factor 2, multiple EGF like domain 11 (MEGF11), erythropoietin (EPO), pigment epithelium-derived factor (PEDF), interleukin-17D (IL17D), interleukin-37 (IL37), interleukin-17C, interleukin-13, interleukin-2, retinol-binding protein 4 (RBP4), tissue factor pathway inhibitor (TFPI2), nerve growth factor beta polypeptide (NGFB), growth hormone somatotropin (GH1/GH), multiple EGF like domain 6 (MEGF6), tumor necrosis factor (TNF), cardiotrophin like cytokine factor 1 (CLCF1), teratocarcinoma-derived growth factor (TDGF), glycoprotein hormone subunit beta 5 (GPHB5), cytokine receptor like factor 1 (CRLF1), transforming growth factor beta (TGFB), growth hormone exon 5 (GHE5), C—C motif chemokine ligand 21 (CCL21), platelet derived growth factor (PDGF), platelet derived growth factor receptor like (PDGFRL), growth differentiation factor (GDF), fibroblast growth factor (FGF), C—X—C motif chemokine ligand (CXCL), and insulin like growth factor binding protein (IGFBP).

6. The method of claim 5, wherein the hormone is insulin cell is a stem cell.

7. The method of claim 1, wherein the stimulating comprises producing more cell division cycles of the cell compared to a cell not contacted with the composition.

8. The method of claim 1, wherein the stimulating comprises which is for prolonging a lifespan of the cell.

9. The method of claim 1, wherein the contacting the cell is in a cell culture.

10. (canceled)
